(19) United States
(12) Patent Application Publication
(10) Pub. No.: US 2011/0269805 A1
(43) Pub. Date: Nov. 3, 2011

(54) METHOD OF TREATING SENSORIMOTOR DISORDERS WITH 4-(1-(2,3-DIMETHYLPHENYL)ETHYL)-1H-IMIDAZOLE-2(3H)-THIONE

(76) Inventors: Daniel W. Gil, Corona Del Mar, CA (US); John E. Donello, Dana Point, CA (US); Lauren M.B. Luhrs, Rancho Santa Margarita, CA (US)

(21) Appl. No.: 12/680,718

(22) PCT Filed: Oct. 14, 2008
(86) PCT No.: PCT/US08/79768

§ 371 Date: Mar. 29, 2010

Related U.S. Application Data
(60) Provisional application No. 60/981,013, filed on Oct. 18, 2007.

(51) Int. Cl. A61K 31/4164; A61P 25/30; A61P 25/36; A61P 25/00; A61P 25/18

(52) U.S. Cl. 514/392

(57) ABSTRACT

Disclosed herein is a method of treating sensorimotor disorders comprising administering to a subject in need of such treatment 4-(1-(2,3-dimethylphenyl)ethyl)-1H-imidazole-2(3H)-thione.

### CROSS-REFERENCE

This application claims the benefit of U.S. Application Ser. No. 60/981,013, filed Oct. 18, 2007, which is hereby incorporated by reference in its entirety.

Disclosed herein is a method of treating sensorimotor disorders by administering to a subject 4-(1-(2,3-dimethylphenyl)ethyl)-1H-imidazole-2(3H)-thione.

### DETAILED DESCRIPTION OF THE INVENTION

### 4-(1-(2,3-dimethylphenyl)ethyl)-1H-imidazole-2(3H)-thione

The compound of the invention, 4-(1-(2,3-dimethylphenyl)ethyl)-1H-imidazole-2(3H)-thione, is described in U.S. Pat. No. 7,141,597, the contents of which are incorporated by reference herein.

The compound is an alpha-2 adrenergic receptor agonist having the following structure:

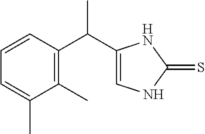

It occurs as two enantiomers. The R-enantiomer is depicted here on the left; the S-enantiomer on the right:

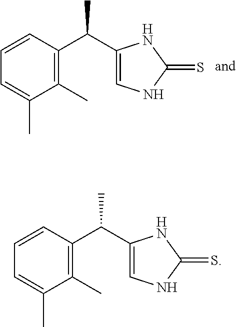

One can use in the methods of the invention either enantiomer, or a mixture of both, as well as any pharmaceutically acceptable salt or prodrug of these compounds.

### Pharmaceutically Acceptable Salts

The compound of the invention may be used as its pharmaceutically acceptable salt.

A “pharmaceutically acceptable salt” is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

### Prodrugs

One can use in the methods of the invention a prodrug of the compound of 4-(1-(2,3-dimethylphenyl)ethyl)-1H-imidazole-2(3H)-thione.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester may be derived from a carboxylic acid of C1 (i.e., the terminal carboxylic acid of a natural prostaglandin), or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

4-(1(2,3-dimethylphenyl)ethyl)-1H-imidazole-2(3H)-thione may be either synthetically produced, or may be produced within the body after administration of a prodrug. Hence, “4-(1-(2,3-dimethylphenyl)ethyl)-1H-imidazole-2(3H)-thione” encompasses both compounds produced by a manufacturing process and those compounds formed in vivo only when another drug administered.

### Dose

The precise dose and frequency of administration depends on the severity and nature of the patient's condition, on the manner of administration, on the potency and pharmacodynamics of the particular compound employed, and on the judgment of the prescribing physician. Determining dose is a routine matter that is well within the capability of someone of ordinary skill in the art. In general, the compound of the invention is administered in therapeutically effective doses, that is, at a dose that is sufficient to produce the desired therapeutic effect.

### Excipients and Dosage Forms

Those skilled in the art will readily understand that the compound of the invention can be admixed with pharmaceutically acceptable excipients which are well known in the art.

A pharmaceutical composition to be administered systemically may be confected as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. No. 4,256,108, No. 4,166,452, and No. 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

### Sensorimotor Disorders

4-(1-(2,3-dimethylphenyl)ethyl)-1H-imidazole-2(3H)-thione is useful in treating sensorimotor disorders. To “treat,” as used here, means to deal with medically. It includes administering the compound of the invention to prevent the onset of a condition, to diminish its severity, and to prevent its reoccurrence. The inventors have discovered that the compound of the invention may be used to treat sensorimotor disorders without causing the sedation that ordinarily accompanies the administration of alpha-2 agonists.

A “sensorimotor disorder” is any condition characterized by abnormal motor output in response to sensory input information. Such disorders are caused by a deficit in sensorimotor gating, the ability of the central nervous system to process sensory input information. Sensorimotor disorders are therefore distinguished from other movement disorders by their cause: a deficit in processing sensory input information creates an urge to perform a motion, whereas in other disorders, movement arises independently of any urges to perform them or other consequences of sensorimotor gating. In this sense, the movements of sensorimotor disorders may not be strictly involuntary, but, rather, merely difficult to suppress. For the purposes of this invention such movements need only interfere with a patient's normal functioning or otherwise be undesirable.

Sensorimotor disorders include or are associated with the following, for example: Tourette's syndrome, transient tic disorder, trichotillomania, attention deficit/hyperactivity disorder (combined type, predominantly hyperactive-compulsive type, predominantly inattentive type, and not otherwise specified (“NOS”)), amphetamine-induced disorders (anxiety, mood and NOS), cocaine-induced disorders (anxiety, mood and NOS), PCP-induced disorders (anxiety, mood and NOS), other (or unknown) substance-induced disorders (anxiety, mood and NOS), post-traumatic stress disorder, autism, and psychoses, such as schizophrenia and other conditions characterized by hallucinations and delusions.

In one embodiment of the invention, a sensorimotor disorder is further characterized by changes (for example, an increase or a decrease) in the availability or utilization of dopamine in the nervous system; hence, the compound of the invention may be used to treat sensorimotor disorders in which hyper- or hypo-dopamine conditions play a role in the etiology of the disorder

In one embodiment of the invention, a sensorimotor disorder is further characterized by defects in prepulse inhibition. In normal subjects, a startle reflex induced by a particular stimulus (pulse) is reduced when the stimulus is preceded by a milder stimulus (prepulse). In a subject with a sensorimotor disorder, the prepulse does not have this effect or its effects are diminished. Prepulse inhibition is a highly validated task that is commonly found to be deficient in various neuropsychiatric disorders such as Tourette's syndrome, schizophrenia, autism, and attention deficit-hyperactivity disorder. A subject experiencing undesired movements who has deficiencies in prepulse inhibition may be presumed to have a sensorimotor disorder.

Methods of assessing prepulse inhibition in human patients is well known in the art. One method, for example, is described in detail in D. L. Braff et al., American Journal of Psychiatry, 156:4 (1999). According to this method, blinking, a component of the startle response, is measured by electromyogram activity transmitted via electrodes positioned over the orbicularis oculi muscle. Subjects are acclimated for five minutes against a continuing background of 70 dB[A] continuous SPL broadband noise, and then proceed to two blocks of trials, with each trial being performed six times, as shown in Table 1.

TABLE 1
experimental protocol for assessing prepulse inhibition. The
startle pulse is 40 msec of 118-dB[A] SPL bursts of noise, and
the prepulse is given as dB[A] above the 70 dB[A] background.
Each trial is separated by 15 seconds.

| BLOCK | TRIAL | MEASURE |
|---|---|---|
| 1 | 1-6 | Startle pulse alone |
| | 7-12 | 2-dB[A] prepulse followed by startle pulse |
| | 13-18 | 4-dB[A] prepulse followed by startle pulse |
| | 19-24 | 8-dB[A] prepulse followed by startle pulse |
| | 25-30 | 16-dB[A] prepulse followed by startle pulse |
| | | No stimulus |
| 2 | 1-6 | Startle pulse alone |
| | 7-12 | 2-dB[A] prepulse followed by startle pulse |
| | 13-18 | 4-dB[A] prepulse followed by startle pulse |
| | 19-24 | 8-dB[A] prepulse followed by startle pulse |
| | 25-30 | 16-dB[A] prepulse followed by startle pulse |

The compounds of the invention may be used to treat any individual, presenting with undesired movements, who shows deficiencies in prepulse inhibition compared to normal subjects tested pursuant to the above protocol.

### Examples

The invention is illustrated by the following examples. This is provided for illustration only; many more embodiments are possible.

### Examples

The invention is illustrated by the following examples. This is provided for illustration only; many more embodiments are possible.

### Amphetamine-Induced Stereotypy and Pre-Pulse Inhibition of the Startle Response

Amphetamine-induced stereotypy is a model of increased dopamine-mediated perseverative behaviors. In this model, the compound of the invention was able to effectively inhibit stereotypy associated with high-dose psychostimulant administration. The compound of the invention was also evaluated in the pre-pulse inhibition of the startle response task (PPI), and found to significantly rescue deficits in sensorimotor integration induced by the psychostimulant amphetamine or phencyclidine.

Importantly, the compound of the invention is orally active, and therefore could be administered in solution, tablet or capsule.

### 4-(1-(2,3-dimethylphenyl)ethyl)-1H-imidazole-2(3H)-thione selectively inhibits amphetamine-induced stereotypy

C57B/6 male mice were placed in an open field apparatus and allowed to habituate for 15-30 minutes. The compound of the invention (at 30 μg/kg) was administered PO at 0, +15, or +30 minutes relative to amphetamine (8 mg/kg) administration, and locomotor behavior was scored for an additional 60 minutes post-amphetamine. Amphetamine was administered IP. Locomotion was separated into “fine movements” (indicative of stereotypy) and “ambulations” (indicative of hyperactivity).

The compound of the invention selectively decreased amphetamine-induced stereotypy, as shown in the following table.

TABLE 2

| | TIME POST- | | |
|---|---|---|---|
| | AMPHE- | | |
| | TAMINE | FINE | |
| GROUP | (MIN) | MOVEMENTS | AMBULATIONS |
| Vehicle + Vehicle | | 740.3 ± 59.5 | 3633.2 ± 946.6 |
| Amphetamine + | | 3375.2 ± 323.4 | 7428.2 ± 577.4* |
| Vehicle | | | |
| AGN-203818 + | −15 | 904.5 ± 107.0^{⋄} | 4756.3 ± 1208.8 |
| Amphetamine | 0 | 762.2 ± 89.4^{⋄} | 7337 ± 890.0* |
| | 15 | 1066.5 ± 37.8^{⋄} | 5747.3 ± 874.4* |
| | 30 | 1297.8 ± 85.3^{⋄} | 7201.2 ± 1669.4* |

^{⋄}indicates significant difference relative to the respective vehicle + amphetamine group.
*indicates significant difference relative to the vehicle + vehicle group.

### 4-(1-(2,3-dimethylphenyl)ethyl)-1H-imidazole-2(3H)-thione restore PPI disrupted by psychostimulants

Male Sprague-Dawley rats were treated with Drug 1 (IP) followed 20 minutes later by Drug 2 (IP or SC as indicated in the methods). Ten minutes following administration of Drug 2 (except in the case of apomorphine where animals were immediately tested post-apomorphine), rats were placed in the startle chamber and allowed 5 minutes to acclimate with 65-dB background white noise. The acclimation period was followed by a ˜15 minute test session during which time rats were presented with 40 ms 120 dB startle pulses alone or preceded 100 ms by a pre-pulse 3, 6, or 12 dB above background. These four types of active stimuli were presented in pseudorandom order along with no-stimulation trials to assess baseline activity throughout testing. An average of 20 s separated each trial type. The maximum startle magnitude was measured for every trial type. PPI for each animal was calculated as the percentage startle magnitude to the pre-pulse+pulse or no-stimulation trials relative to the pulse-alone startle magnitude.

TABLE 3
the compound of the invention (0.01, 0.03, 0.1, and 1 mg/kg)
rescues PPI disruption following amphetamine or phencyclidine (PCP)
administration. Clonidine (30 ug/kg) was used as a positive control.

| | | % PPI | | |
|---|---|---|---|---|
| DRUG 1 | DRUG 2 | PP3 | PP6 | PP12 |
| Vehicle | Vehicle | 47.8 ± 5.1% | 58.2 ± 3.6% | 60.0 ± 4.0% |
| Vehicle | Amphetamine | 16.7 ± 6.3%* | 35.8 ± 3.9%* | 44.8 ± 3.7%* |
| | | 27.7 ± 3.2%* | | |
| Vehicle | PCP | | 29.2 ± 12.6%* | 49.6 ± 8.4% |
| Clonidine | Vehicle | 35.2 ± 15.7% | 60.8 ± 3.0%^{⋄} | 54.7 ± 8.0% |
| Clonidine | Amphetamine | 31.0 ± 6.9% | 40.7 ± 6.0%* | 53.5 ± 5.2% |
| Clonidine | PCP | 34.5 ± 8.2% | 55.1 ± 9.8% | 63.7 ± 7.4% |
| Compound | Vehicle | 60.4 ± 4.8%^{⋄} | 59.8 ± 2.2%^{⋄} | 75.5 ± 2.9%^{⋄}* |
| of the invention | | | | |
| Compound | Amphetamine | 49.6 ± 5.8%^{⋄} | 52.9 ± 4.3%^{⋄} | 65.7 ± 4.7%^{⋄} |
| of the invention (1) | | | | |
| Compound | PCP | 20.3 ± 10.6% | 21.6 ± 10.7% | 51.8 ± 5.6% |
| of the invention | | | | |
| (0.01) | | | | |
| Compound | PCP | 30.9 ± 7.4% | 46.2 ± 4.2%^{⋄} | 53.6 ± 4.2% |
| of the invention | | | | |
| (0.03) | | | | |
| Compound | PCP | 35.8 ± 7.0% | 39.6 ± 10.7% | 56.2 ± 5.6%^{⋄} |
| of the invention | | | | |
| (0.1) | | | | |
| Compound | PCP | 48.8 ± 8.2%^{⋄} | 45.0 ± 12.7% | 60.5 ± AA8.1%^{⋄} |
| of the invention (1) | | | | |

^{⋄}indicates significant difference relative to the respective vehicle + psychostimulant group.
*indicates significant difference relative to the vehicle + vehicle group.

### Methods

Amphetamine induced stereotypy. C57B/6 male mice were placed in an open field apparatus and allowed to habituate for 15-30 minutes. 4-(1-(2,3-dimethylphenyl)ethyl)-1h-imidazole-2(3h)-thione was administered p.o. at −15, 0, +15, or +30 minutes relative to amphetamine (8 mg/kg, s.c.) administration and locomotor behavior was scored for an additional 60 minutes post-amphetamine. Locomotion was separated into “fine movements” (indicative of stereotypy) and “ambulations” (indicative of hyperactivity).
Prepulse inhibition. Sprague Dawley male rats (˜200 g-300 g) were used (n=8/group). Rats were treated with vehicle, clonidine (0.03 mg/kg, s.c.) or 4-(1-(2,3-dimethylphenyl)ethyl)-1h-imidazole-2(3h)-thione (0.01, 0.03, 0.1 or 1 mg/kg, s.c.) followed by phencyclidine (2 mg/kg, s.c.), amphetamine (4 mg/kg, s.c.) or vehicle 20 minutes later. Each rat was individually placed in the startle chamber 10 minutes post-phencyclidine or post-amphetamine or post-vehicle. Testing was carried out using the SR-Lab Startle Response system by San Diego Instruments. Each rat was allowed to acclimate for a 5 minute period with a 65-dB background noise continuously present throughout the session. The acclimation was followed by a 15 minute PPI test session where rats were presented with 120 dB startle pulses without a pre-pulse or pulses preceded by a pre-pulse of 3, 6, or 12 dB above background noise. These active stimuli were presented in pseudorandom order along with no-sound trials with an average of 20 sec separating them. A sensor in the chamber recorded the startle responses following all stimuli presented. PPI data were calculated as a percentage of PPI by comparing the pulse-alone versus the pre-pulse trials and are presented as % PPI for each pre-pulse intensity. 441-(2,3-dimethylphenyl)ethyl)-1h-imidazole-2(3h)-thione was administered i.p. and amphetamine and phencyclidine were administered s.c.

What is claimed is:

1. A method of treating a sensorimotor disorder comprising administering to a subject in need of such treatment a compound having the following structure:

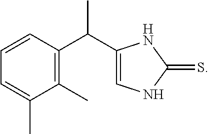

2. The method of claim 1, wherein the method further comprises treating the sensorimotor disorder without causing sedation.

3. The method of claim 2, wherein the sensorimotor disorder is associated with a hyper- or hypo-dopamine condition of the nervous system.

4. The method of claim 2, wherein the subject has a deficiency in prepulse inhibition.

5. The method of claim 2, wherein the disorder is selected from or associated with the following disorders: Tourette's syndrome, transient tic disorder, trichotillomania, attention deficit/hyperactivity disorder, amphetamine-induced disorders, cocaine-induced disorders, PCP-induced disorders, other (or unknown) substance-induced disorders, post-traumatic stress disorder, autism, and psychosis.
